(19) United States
(12) Patent Application Publication
(10) Pub. No.: US 2023/0364356 A1
(43) Pub. Date: Nov. 16, 2023

(54) Fixing Assembly

(71) Applicant: Merxin Ltd, King's Lynn (GB)

(72) Inventor: Adam Stuart, King's Lynn (GB)

(21) Appl. No.: 17/745,364

(22) Filed: May 16, 2022

(51) Int. Cl. A61M 11/02

(52) U.S. Cl. CPC A61M 11/02

(57) ABSTRACT

In general terms the present invention proposes a nozzle fixing assembly 100 for an inhalation device 10. The nozzle fixing assembly 100 comprises a nozzle 110 enclosed between a nozzle holder 122 and a chamber body 150. The nozzle holder 122 provides a holder abutment surface 129 which abuts at least a portion of an abutment surface 152 of the chamber body 150 to define an abutting interface 170. A nozzle seal 180 is arranged annularly around at least a portion of the nozzle 110 and extends between the nozzle 110 and the nozzle holder 122 and between the nozzle 110 and the chamber body 150 such that the nozzle seal 180 makes sealing contact with the nozzle holder 122 and chamber body 150 and provides a perimetral line of sealing at the abutting interface 170.

### TECHNICAL FIELD

This invention relates to the nebulisation of liquids. In particular, though not exclusively, this invention relates to a nozzle fixing assembly for an inhalation device and to an inhalation device comprising the nozzle fixing assembly.

### BACKGROUND

Drug delivery devices such as soft mist inhalers (SMIs) can be used to produce an aerosol of droplets for inhalation through the mouth and pharyngeal cavity into the lungs of a patient, for nasal administration, or for spraying the surface of the eye.

In an inhaler of this kind, liquid pharmaceutical formulations are typically stored in a reservoir. From there, they are conveyed through a riser tube into a pressure chamber from where they are forced through a nozzle under pressure and atomised. In this way, SMIs are able to nebulise a small amount of a liquid formulation according to the required dosage within a few seconds to produce an aerosol suitable for therapeutic inhalation. Moreover, this can be achieved without requiring the use of a propellant.

The nozzle is typically held in place in the device by a seal and fixing means. As the device is actuated, liquid formulation is forced through the nozzle under high pressures. These high pressures are significant enough to extrude seal material through any small gaps in the componentry and are high enough to damage the structure of the nozzle or result in failure of the seal.

Hence, there is a need for a seal configuration which minimises the pressure differential between the inside and the outside of the nozzle and is configured in such a way that seal extrusion is minimised and the excess pressures inside the sealing configuration during actuation serve to improve the seal on the nozzle configuration. It is an object of the invention to address at least one of the above problems, or another problem associated with the prior art.

### SUMMARY OF THE INVENTION

A first aspect of the invention provides a nozzle fixing assembly for an inhalation device. The nozzle fixing assembly comprises a nozzle having an inlet end and an outlet end, the outlet end being distal with respect to the inhalation device and configured to deliver a liquid for inhalation, the inlet end being proximal with respect to the inhalation device and being configured to receive the liquid for inhalation.

The nozzle fixing assembly also comprises a nozzle holder comprising a holder portion that encircles at least a portion of the outlet end of the nozzle and an end wall with an aperture, such that the end wall retains the nozzle, and the aperture allows delivery of the liquid from the outlet end of the nozzle, the holder portion comprising a holder abutment surface facing proximally.

The nozzle fixing assembly further comprises a chamber body which is proximal with respect to the nozzle holder and defines a chamber communicating with the inlet end of the nozzle to supply the liquid to the nozzle, the chamber body comprising a chamber portion encircling at least a proximal portion of the chamber, and comprising a first chamber abutment surface that faces distally and abuts at least a portion of the holder abutment surface to define an abutting interface.

The nozzle fixing assembly also comprises a nozzle seal arranged annularly around at least a portion of the nozzle to make sealing contact with the nozzle. The nozzle seal has a first end that at least partially abuts the end wall of the nozzle holder, and a second end at or towards the inlet end of the nozzle. The nozzle seal extends between the nozzle and the nozzle holder and between the nozzle and the chamber body such that the nozzle seal makes sealing contact with the nozzle holder and chamber body and provides a perimetral line of sealing at the abutting interface.

The chamber body comprises a second chamber abutment surface facing distally. An annularly outer portion of the second end of nozzle seal makes sealing contact with the second chamber abutment surface. An annularly inner portion of the second end of the nozzle seal is recessed such that the chamber extends into the second end of the nozzle seal.

In this way, a liquid entering the chamber under pressure may advantageously exert a force on the annularly inner portion of the second end of the nozzle seal, which may force the seal against the nozzle holder and chamber body and provide a particularly tight perimetral line of sealing at the abutting interface. Moreover, excess pressures inside the nozzle fixing assembly may serve to improve the radial sealing between the nozzle holder and nozzle seal and between the chamber body and nozzle seal. Significantly, this may reduce any leakage of the liquid from the nozzle fixing assembly.

Additionally, due to the chamber extending into the second end of the nozzle seal, the chamber may advantageously surround a portion of a side or sides of the nozzle. Thus, this may allow the liquid entering the chamber under pressure to exerting a force directly on one or more sides of the nozzle. This may advantageously minimise the pressure differential between the inside and the outside of the nozzle, thereby preventing damage to the nozzle.

Suitably, the liquid may be a pharmaceutical liquid. The term “pharmaceutical liquid” as defined herein refers to a solution, emulsion, or suspension of one or more active pharmaceutical ingredients in a suitable solvent.

In some embodiments, the nozzle may comprise a nozzle chip. A “nozzle chip” as defined herein is a component having an inlet end and an outlet end connected by a plurality of microstructured channels. The inlet end of the nozzle chip may comprise a filtering structure, comprising one or more microstructured channels that are generally zig-zag shaped (i.e. form a generally zig-zag structure). In this way, the filtering structure may advantageously prevent any coarse debris from blocking the microstructured channels connecting the inlet and outlet ends of the nozzle chip. The outlet end of the nozzle chip may comprise one or more spray jets. Where two or more spray jets are present, the geometries of the spray jets may be suitably be arranged to cause two or more jets of liquid exiting the spray jets to impinge upon one another (i.e. collide with each other).

In some embodiments, the annularly inner portion of the nozzle seal may be recessed to comprise a nozzle facing surface facing the nozzle and a proximal facing inner surface facing the inhalation device, meeting at a sharp edge, defining a recess which extends circumferentially around at least a portion the nozzle. For example, the nozzle facing surface and the proximal facing inner surface may meet at an angle of about 40°, or of about 50°, or of about 60°, or of about 70°, or of about 80°, or of about 90°, or of about 100°, or of about 110°, or even of about 120°. Suitably, the nozzle facing surface and the proximal facing inner surface may meet at an angle of about 85°, or of about 86°, or of about 87°, or of about 88°, or of about 89°, or of about 91°, or of about 92°, or of about 93°, or of about 94°, or even of about 95°.

In some embodiments, the nozzle seal may be recessed to define a cylindrical recess having a sharp angled inner circumferential edge. For example, the inner circumferential edge may define a sharp angle of about 40°, or of about 50°, or of about 60°, or of about 70°, or of about 80°, or of about 90°, or of about 100°, or of about 110°, or even of about 120°. Suitably, the inner circumferential edge may define a sharp angle of about 85°, or of about 86°, or of about 87°, or of about 88°, or of about 89°, or of about 91°, or of about 92°, or of about 93°, or of about 94°, or even of about 95°.

In some embodiments, the annularly inner portion of the nozzle seal may be recessed to comprise a nozzle facing surface facing the nozzle and a proximal facing inner surface facing the inhalation device, meeting at a radiused edge, defining a recess which extends circumferentially around at least a portion of the nozzle.

In some embodiments, the nozzle seal may be recessed to define a cylindrical recess having a radiused inner circumferential edge.

In some embodiments, the annularly inner portion of the nozzle seal may be recessed to comprise a nozzle facing surface facing the nozzle and a proximal facing inner surface facing the inhalation device, meeting at a chamfered edge, defining a recess which extends circumferentially around at least a portion of the nozzle.

In some embodiments, the nozzle seal may be recessed to define a cylindrical recess having a chamfered inner circumferential edge.

In some embodiments, the annularly inner portion of the nozzle seal may be recessed to comprise a nozzle facing surface facing the nozzle and a proximal facing inner surface facing the inhalation device, meeting at a filleted edge, defining a recess which extends circumferentially around at least a portion of the nozzle.

In some embodiments, the nozzle seal may be recessed to define a cylindrical recess having a filleted inner circumferential edge.

A second aspect of the invention provides an inhalation device comprising a nozzle fixing assembly according to a first aspect of the invention.

The inhalation device may suitably be an inhaler for nebulising pharmaceutical liquids. For example, the inhalation device may suitably be a soft mist inhaler (SMI).

Throughout the description and claims of this specification, the words “comprise” and “contain” and variations of the words, for example “comprising” and “comprises”, mean “including but not limited to”, and do not exclude other components, integers or steps. Moreover, the singular encompasses the plural unless the context otherwise requires: in particular, where the indefinite article is used, the specification is to be understood as contemplating plurality as well as singularity, unless the context requires otherwise.

Preferred features of each aspect of the invention may be as described in connection with any of the other aspects. Within the scope of this application it is expressly intended that the various aspects, embodiments, examples and alternatives set out in the preceding paragraphs, in the claims and/or in the following description and drawings, and in particular the individual features thereof, may be taken independently or in any combination. That is, all embodiments and/or features of any embodiment can be combined in any way and/or combination, unless such features are incompatible.

### BRIEF DESCRIPTION OF THE DRAWINGS

One or more embodiments of the invention will now be described, by way of example only, with reference to the accompanying drawings, in which:

FIG. 1 is a cross sectional view of a nozzle fixing assembly in accordance with a first embodiment of the invention;

FIG. 2 is a close up of the cross sectional view of the nozzle fixing assembly shown in FIG. 1;

FIG. 3 is a perspective view of the nozzle seal shown in the nozzle fixing assembly in FIGS. 1 and 2;

FIG. 4 is a cross sectional view of a nozzle fixing assembly in accordance with a second embodiment of the invention; and

FIG. 5 is a perspective view of the nozzle seal shown in the nozzle fixing assembly in FIG. 4.

### DETAILED DESCRIPTION

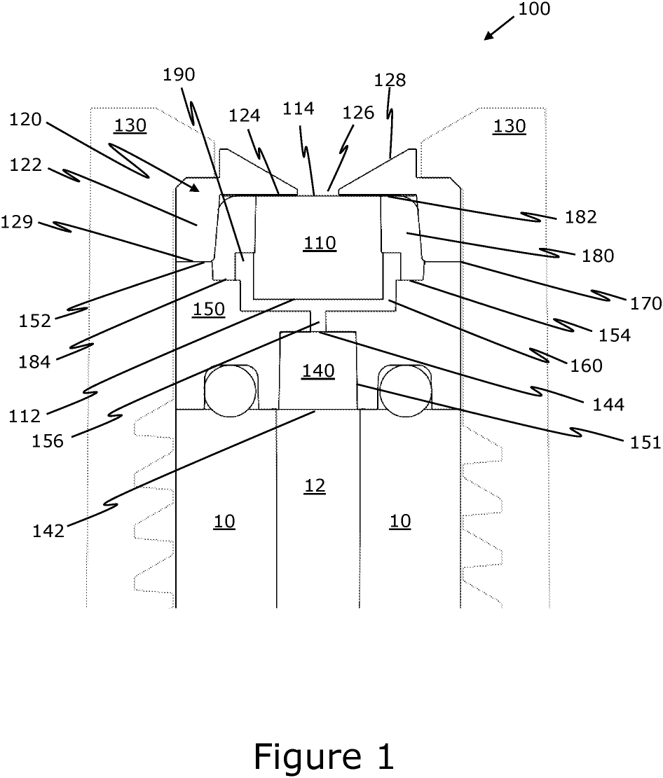

Referring to FIG. 1, a nozzle fixing assembly 100 in accordance with a first embodiment of the invention has a nozzle chip 110 having an inlet end 112 and an outlet end 114. The inlet end 112 faces proximally with respect an inhalation device 10 in which the nozzle fixing assembly 100 is incorporated. The outlet end 114 faces distally with respect the inhalation device 10. The nozzle fixing assembly 100 also comprises a nozzle holder 120, which has a holder portion 122 that surrounds the outlet end 114 of the nozzle chip 110 circumferentially to about halfway along the nozzle chip 110 between its inlet and outlet ends 112, 114.

The nozzle holder 120 has an end wall 124 that faces proximally with respect to the inhalation device and retains the outlet end 114 of the nozzle 110. An aperture 126 is arranged in the centre of the end wall 124 to allow the delivery of liquid from the outlet end 114 of the nozzle 110. The nozzle holder 120 also has a conically shaped recess 128, which surrounds the aperture 126 and faces distally with respect to the inhalation device 10 on the opposite side of the nozzle holder 122 to the end wall 124. The holder portion 122 has a circumferential nozzle holder abutment surface 129 which faces proximally with respect to the inhalation device 10.

The nozzle fixing arrangement 100 comprises a nut 130 for fixing the nozzle holder 110 in place in the inhalation device 10. The nozzle fixing arrangement 100 also has a filter 140 having an inlet end 142 and an outlet end 144. The filter 140 is held in place inside the inhalation device 10 by a filter holder 150. The filter holder 150 has a distally facing side which faces the inlet end 122 of the nozzle chip 110, and a proximally facing side which faces the inhalation device 10. The filter 140 is accommodated by a recess 151 in the centre of the proximally facing side of the filter holder 150.

An annularly inner portion of the distally facing side of the filter holder 150 is recessed to define a chamber 160 between the filter holder 150 and the inlet end 122 of the nozzle chip 110. The chamber 160 surrounds the inlet end 122 and extends annularly around the nozzle chip 110 to about halfway along the nozzle chip 110 between its inlet and outlet ends 112, 114. A channel 156 passes through the centre of the filter holder 150 between the chamber 160 and the recess 151, bringing the chamber 160 into fluid connection with the outlet end 144 of the filter 140. The inlet end 142 of the filter 140 is in fluid connection with a conduit 12 in the inhalation device 10.

An annularly outer portion of the distally facing side of the filter holder 150 defines a first filter holder abutment surface 152 which faces distally with respect to the inhalation device 10. The distally facing first filter holder abutment surface 152 mates with the proximally facing nozzle holder abutment surface 129 to define an abutting interface 170 between the first filter holder abutment surface 152 and the nozzle holder abutment surface 129. In this way, the nozzle 110 and chamber 160 are enclosed between the nozzle holder 120 and filter holder 150.

A nozzle seal 180 is arranged annularly around the nozzle chip 110 and extends from the outlet end 114 of the nozzle chip 110 to about halfway along the nozzle chip 110 between its inlet and outlet ends 112, 114, making sealing contact with the nozzle chip 110. The nozzle seal 180 has a first end 182 which faces distally with respect to the inhalation device 10 and abuts the end wall 124 of the nozzle holder 120. The nozzle seal 180 has a second end 184 on the opposite side of the nozzle seal 180 to the first end 182 which faces proximally with respect to the inhalation device 10. A second filter holder abutment surface 154 arranged annularly between the chamber 160 and the first filter holder abutment surface 152 faces distally with respect to the inhalation device 10 and abuts an annularly outer portion second end 184 of the nozzle seal 180.

The nozzle seal 180 extends between the nozzle chip 110 and the holder portion 122 and between the nozzle chip 110 and the filter holder 150 such that the nozzle seal 180 makes sealing contact with the holder portion 122 and filter holder 150 and provides a perimetral line of sealing at the abutting interface 170 between the first filter holder abutment surface 152 and the nozzle holder abutment surface 129. An annularly inner portion of the second end 184 of the nozzle seal 180 is recessed such that the chamber 160 extends into the second end 184 of the nozzle seal 180. In this example, the annularly inner portion of the second end 184 of the nozzle seal 180 is recessed to define a nozzle facing surface 186 and a proximal facing inner surface 187. The nozzle facing surface 186 and proximal facing inner surface 187 meet at a 90° angle to form a sharp edge 188 and define a recess 190 which extends circumferentially around the nozzle chip 110.

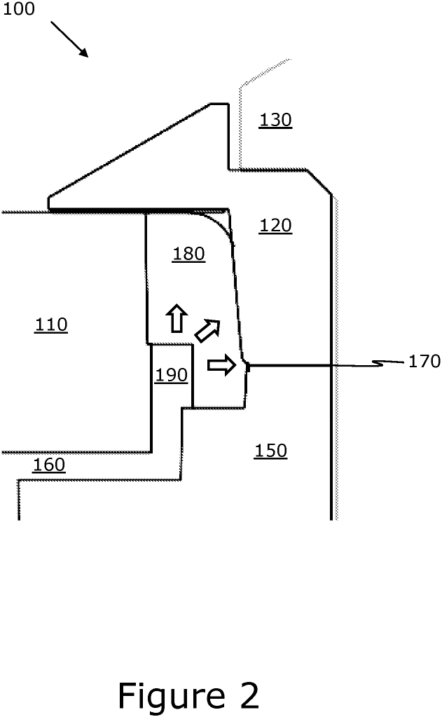

FIG. 2 provides a close up of the cross sectional view of the nozzle fixing assembly 1oo shown in FIG. 1. FIG. 2 has been marked up with arrows which show the direction of the force exerting on the nozzle seal 180 by liquid in the recess 190 when the inhalation device 10 is in use. Pressure exerted on the nozzle seal 180 by the high pressure liquid in the recess 190 enhances the seal between nozzle seal 180 and the nozzle holder 120 and between the nozzle seal 180 and the filter holder 150. This improves the radial sealing between the nozzle holder 120 and nozzle seal 180 and between the filter holder 150 and nozzle seal 180 and reduces any leakage of the liquid from the nozzle fixing assembly 100.

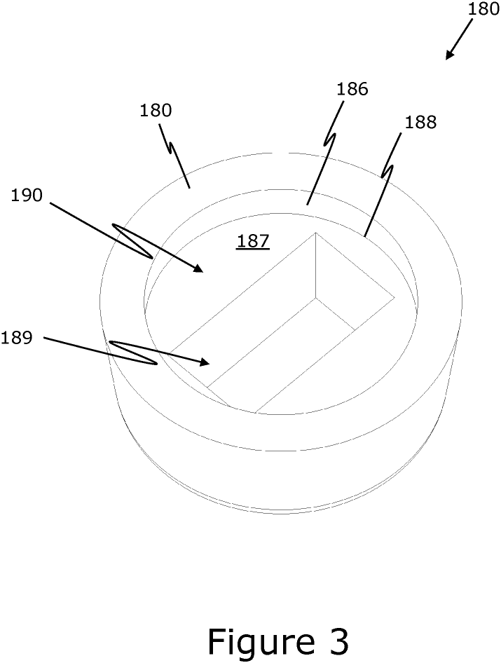

FIG. 3 provides a perspective view of the nozzle seal 180. As mentioned above, the annularly inner portion of the second end 184 of the nozzle seal 180 is recessed to define a nozzle facing surface 186 and a proximal facing inner surface 187. The nozzle facing surface 186 and proximal facing inner surface 187 meet at a 90° angle to form a sharp edge 188 and define a recess 190 which extends circumferentially around the nozzle chip 110. The proximal facing inner surface 187 of the nozzle seal 180 also comprises a rectangular slot 189 for accommodating the nozzle chip 190.

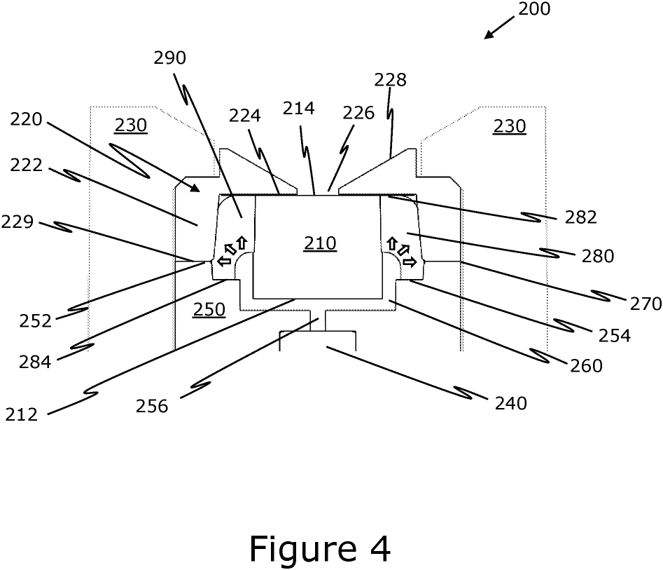

FIG. 4 illustrates a nozzle fixing assembly 200 in accordance with a second embodiment of the invention The nozzle fixing assembly 200 has generally the same structure as the nozzle fixing assembly 100 in accordance with a first embodiment of the invention, the exception being that the annularly inner portion of the second end 284 of the nozzle seal 280 is recessed to define a nozzle facing surface 286 and a proximal facing inner surface 287, which meet to form a chamfered edge 288. FIG. 4 has been marked up with arrows which show the direction of the force exerting on the nozzle seal 280 by high pressure liquid in the recess 290 when the inhalation device 10 is in use. The chamfered edge 288 of the recess 290 may help to provide a more distributed pressure profile through the nozzle seal 280.

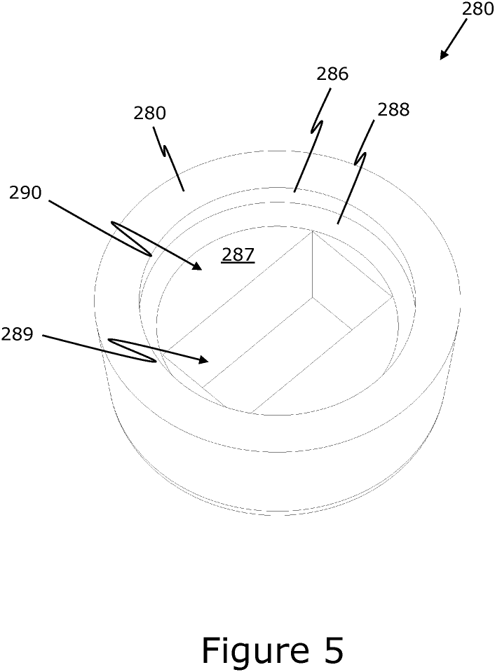

FIG. 5 provides a perspective view of the nozzle seal 280. As mentioned above, the annularly inner portion of the second end 284 of the nozzle seal 280 is recessed to define a nozzle facing surface 286 and a proximal facing inner surface 287. The nozzle facing surface 286 and proximal facing inner surface 287 meet to form a radiused edge 288 and define a recess 290 which extends circumferentially around the nozzle chip 210. The proximal facing inner surface 287 of the nozzle seal 280 also comprises a rectangular slot 289 for accommodating the nozzle chip 290.

1. A nozzle fixing assembly for an inhalation device comprising:
(i) a nozzle having an inlet end and an outlet end, the outlet end being distal with respect to the inhalation device and configured to deliver a liquid for inhalation, the inlet end being proximal with respect to the inhalation device and being configured to receive the liquid for inhalation;
(ii) a nozzle holder comprising a holder portion that encircles at least a portion of the outlet end of the nozzle and an end wall with an aperture, such that the end wall retains the nozzle and the aperture allows delivery of the liquid from the outlet end of the nozzle, the holder portion comprising a holder abutment surface facing proximally;
(iii) a chamber body which is proximal with respect to the nozzle holder and defines a chamber communicating with the inlet end of the nozzle to supply the liquid to the nozzle, the chamber body comprising a chamber portion encircling at least a proximal portion of the chamber, and comprising a first chamber abutment surface that faces distally and abuts at least a portion of the holder abutment surface to define an abutting interface; and
(iv) a nozzle seal arranged annularly around at least a portion of the nozzle to make sealing contact with the nozzle, wherein the nozzle seal has a first end that at least partially abuts the end wall of the nozzle holder, and a second end at or towards the inlet end of the nozzle, wherein the nozzle seal extends between the nozzle and the nozzle holder and between the nozzle and the chamber body such that the nozzle seal makes sealing contact with the nozzle holder and chamber body and provides a perimetral line of sealing at the abutting interface;
wherein the chamber body comprises a second chamber abutment surface facing distally, and wherein an annularly outer portion of the second end of nozzle seal makes sealing contact with the second chamber abutment surface; and wherein an annularly inner portion of the second end of the nozzle seal is recessed such that the chamber extends into the second end of the nozzle seal.

2. The nozzle fixing assembly of claim 1, wherein the annularly inner portion of the nozzle seal is recessed to comprise a nozzle facing surface and a proximal facing inner surface, meeting at a sharp edge, defining a recess which extends circumferentially around at least a portion the nozzle.

3. The nozzle fixing assembly of claim 1, wherein the annularly inner portion of the nozzle seal is recessed to comprise a nozzle facing surface and a proximal facing inner surface, meeting at a radiused edge, defining a recess which extends circumferentially around at least a portion of the nozzle.

4. The nozzle fixing assembly of claim 1, wherein the annularly inner portion of the nozzle seal is recessed to comprise a nozzle facing surface and a proximal facing inner surface, meeting at a chamfered edge, defining a recess which extends circumferentially around at least a portion of the nozzle.

5. An inhalation device comprising a nozzle fixing assembly according to claim 1.

6. The inhalation device of claim 5, being a soft mist inhaler (SMI).
